(19) United States
(12) Patent Application Publication
(10) Pub. No.: US 2017/0315103 A1
(43) Pub. Date: Nov. 2, 2017

(54) APPARATUS AND METHOD FOR DEALING WITH AMBIENT SMELL

(71) Applicant: Nokia Technologies Oy, Espoo (FI)

(72) Inventors: Debmalya BISWAS, Lausanne (CH); Matthew John LAWRENSON, Lausanne (CH); Julian NOLAN, Lausanne (CH)

(21) Appl. No.: 15/520,136

(22) PCT Filed: Oct. 31, 2014
(86) PCT No.: PCT/FI2014/050818

§ 371 Date: Apr. 19, 2017

(51) Int. Cl. G01N 33/00; G06F 17/30; G01N 37/00

(52) U.S. Cl. CPC G01N 33/0004; G06F 17/30; G01N 37/005; G01N 33/0009; G01N 33/0031; G01N 33/0036

(57) ABSTRACT

An apparatus comprising: a sensor configured to sense ambient smell; and a processor configured, responsive to the sensor sensing the ambient smell, to determine smell impact based on the ambient smell, and to conduct at least one counter action based on the smell impact.

### TECHNICAL FIELD

The present application generally relates to dealing with ambient smell and in particular, but not exclusively, to dealing with ambient smells that may influence user behaviour.

### BACKGROUND

This section illustrates useful background information without admission of any technique described herein representative of the state of the art.

Purposive use of smell is becoming increasingly popular as a marketing tool to capture user response to different stimuli and to influence customer behaviour. For example some scent emitted in a store may stimulate customers to perform certain actions such as to buy certain products.

### SUMMARY

Various aspects of examples of the invention are set out in the claims.

According to a first example aspect of the present invention, there is provided an apparatus, comprising: a sensor configured to sense ambient smell; and a processor configured, responsive to the sensor sensing ambient smell, to determine smell impact based on the ambient smell, and to conduct at least one counter action based on the smell impact. The apparatus can be an apparatus of a user or a user device. In an example embodiment the apparatus is a portable apparatus/user device or a handheld apparatus/user device.

In an embodiment the counter action comprises recommending a new location to a user of the apparatus. As an example, the counter action may comprise outputting such recommendation to a user of the apparatus for example on a display of the apparatus or as an audible output.

In an embodiment the counter action comprises notifying a user of the apparatus about the smell impact.

In an embodiment the counter action comprises starting active protection. In an example embodiment the active protection is an action against the sensed ambient smell.

In an embodiment the processor is configured to perform the active protection by controlling the apparatus to emit a counter smell to compensate the ambient smell sensed by the sensor.

In an embodiment the determination of the smell impact comprises quantifying a degree of harmfulness of the ambient smell.

In an embodiment the processor is configured to quantify the degree of harmfulness of the ambient smell on the basis of one or more of the following: user sensitivity to the ambient smell, trustworthiness of the provider of the ambient smell, history of smells associated with a location where the ambient smell is sensed, and feedback obtained from other users.

In an embodiment the determination of the smell impact comprises obtaining information about an expected user response to the sensed ambient smell.

In an embodiment the processor is configured to determine user sensitivity to the expected user response.

In an embodiment the processor is configured to give weight to the expected user response on the basis of one or more of the following: user sensitivity to the expected user response, a degree of harmfulness of the ambient smell, trustworthiness of the provider of the ambient smell, history of smells associated with a location where the ambient smell is sensed, and feedback obtained from other users. For example, if user is not very sensitive to the expected user response or the degree of harmfulness of the ambient smell is minimal, the expected user response can be given minimal weighting factor whereby the expected user response is considered to be minimal in the particular case at hand.

In an embodiment the processor is configured to deduct nearby smells from the sensed ambient smell.

In an embodiment the determination of the smell impact comprises taking into account history of smells associated with a location where the ambient smell is sensed. The history of smells can be obtained from a database where information about sensed smells at different locations is collected and stored.

In an embodiment the determination of the smell impact comprises taking into account smell feedback obtained from other users.

The other users associated with the feedback from other users can be users of other similar apparatuses or the feedback can be obtained in some other suitable manner. In an embodiment the feedback from other users and information about sensed smells at certain locations is continuously collected and stored in a database for future use.

In an embodiment the ambient smell is a smell emanated by a user of the apparatus.

In an embodiment the apparatus is a portable user device or a handheld user device.

According to a second example aspect of the present invention, there is provided a method, comprising: sensing ambient smell; and responsive to sensing ambient smell, determining smell impact based on the ambient smell, and conducting at least one counter action based on the smell impact.

In an embodiment the counter action comprises one or more of the following: recommending a new location to a user, notifying a user of the smell impact, and starting active protection.

In an embodiment the active protection comprises emitting a counter smell to compensate the ambient smell.

In an embodiment determining smell impact comprises quantifying a degree of harmfulness of the ambient smell.

In an embodiment determining smell impact comprises obtaining information about an expected user response to the sensed ambient smell, and giving weight to the expected user response on the basis of one or more of the following: user sensitivity to the expected user response, a degree of harmfulness of the ambient smell, trustworthiness of the provider of the ambient smell, history of smells associated with a location where the ambient smell is sensed, and feedback obtained from other users.

In an embodiment determining smell impact comprises one or more of the following: obtaining information about an expected user response to the sensed ambient smell, determining user sensitivity to the expected user response, determining trustworthiness of the provider of the ambient smell, deducting nearby smells from the sensed ambient smell, taking into account history of smells, taking into account smell feedback obtained from other users.

According to a third example aspect of the present invention there is provided a computer program product comprising computer code for causing performing the method of the second example aspect, when executed by an apparatus. Additionally, in further examples, the computer program product comprises computer code for causing performing the method of one or more example embodiments of the invention, when executed by an apparatus.

According to a fourth example aspect of the invention there is provided a non-transitory memory medium comprising the computer program of the third example aspect of the invention.

Any foregoing memory medium may comprise a digital data storage such as a data disc or diskette, optical storage, magnetic storage, holographic storage, opto-magnetic storage, phase-change memory, resistive random access memory, magnetic random access memory, solid-electrolyte memory, ferroelectric random access memory, organic memory or polymer memory. The memory medium may be formed into a device without other substantial functions than storing memory or it may be formed as part of a device with other functions, including but not limited to a memory of a computer, a chip set, and a sub assembly of an electronic device.

Different non-binding example aspects and embodiments of the present invention have been illustrated in the foregoing. The embodiments in the foregoing are used merely to explain selected aspects or steps that may be utilized in implementations of the present invention. Some embodiments and implementation details may be presented only with reference to certain example aspects of the invention. It should be appreciated that corresponding embodiments and implementation details may apply to other example aspects as well.

### BRIEF DESCRIPTION OF THE DRAWINGS

For a more complete understanding of example embodiments of the present invention, reference is now made to the following descriptions taken in connection with the accompanying drawings in which:

FIG. 1 shows an overview of a system of an example embodiment of the invention;

FIG. 2 shows a block diagram of an apparatus according to an example embodiment of the invention; and

FIG. 3 shows a flow diagram illustrating a method according to an example embodiment of the invention.

### DETAILED DESCRIPTION OF THE DRAWINGS

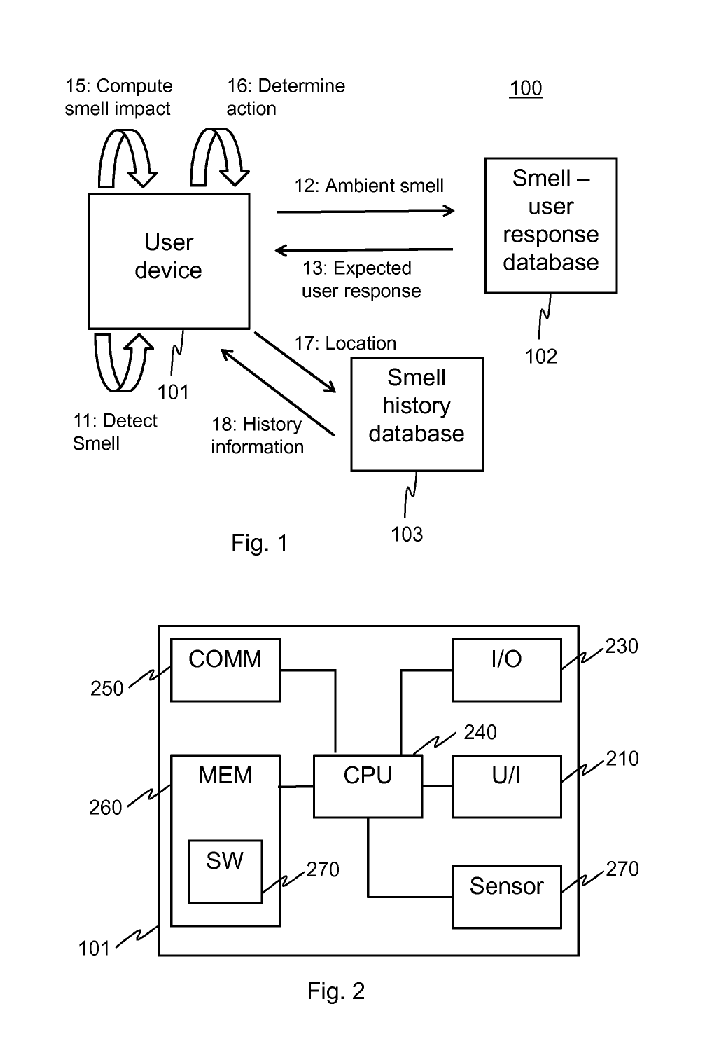

An example embodiment of the present invention and its potential advantages are understood by referring to FIGS. 1 through 3 of the drawings. In this document, like reference signs denote like parts or steps.

As mentioned in background section, ambient smell can be used to influence customer behaviour and to study user response to certain stimuli. As compared to other senses, smell is particularly significant from a privacy perspective as smell is considered to be closely related to emotional reactions. For example a comment by James Viahos outlines “(With) all of the other senses, you think before you respond, but with scent, your brain responds before you think.” Therefore, people do not necessarily notice that they are influenced by artificial ambient smells.

From user perspective ambient smells can cause privacy risks: Covert smells can be used to study the user response to different stimuli, often in a surreptitious fashion—without informing the user and without obtaining explicit consent from the user for the study.

Users cannot be expected to have the requisite knowledge, recall/storage capability, to identify situations associated with potentially malicious smells possibly compromising users' privacy or causing otherwise adverse or unwanted effects to the users. For example performing one or more of the following activities manually may be difficult:
Different users have different reactions to different smells. The research/knowledge regarding the types of reactions a smell can induce in a person is also continuously evolving. A user thus might actually be unaware of the effects of a specific type of smell—when exposed to it.
To determine “maliciousness” of a retailer (or more generally, a provider of ambient smell), there is a need to detect, classify, and remember the types of smells encountered by a user over his past visits to the retailer; possibly co-related with experiences of other users as well. A “malicious” smell might be present by accident as well. To determine malicious intent, ambient smell data—encountered over multiple visits of the user—needs to be collected, classified, and compared.

On the basis of this, there is a need for a system that is configured to take ambient smells into account for example by evaluating impacts ambient smells may have on a user. In particular, there is a need for an automated system for detecting ambient smell and classifying the reactions it can induce in the user. In an example embodiment such automated system is implemented in the user's device, such as a mobile phone, tablet or other handheld or portable electronic device. In an example embodiment the user device co-operates with an updateable database comprising information about different reactions to different smells. The database can be implemented in the user device or it can be a service provided in a server that is accessible over a communication network.

In an example embodiment there is provided an apparatus configured to detect ambient smells and to process information about the ambient smells that are detected. In an embodiment the apparatus is configured to take actions to protect user privacy with respect to ambient smells.

In an example embodiment ambient smell is detected and in response a smell impact quantifying influences the smell may have is determined. The smell impact is then used for determining actions to be taken to deal with the ambient smell.

In an example embodiment it is considered that there is a need to quantify the privacy risk posed by ambient smell to a user and to notify him of the potential privacy risks, possibly accompanied with a recommendation of an appropriate privacy preserving action.

In an example embodiment information about ambient smells is processed in an application running in a user device. The user may turn the application on and off as necessary, whereby the user is able to choose the situations where she wants to use the device for monitoring ambient smells and for protecting her privacy in relation to ambient smells.

FIG. 1 shows an architectural overview of a system 100 of an example embodiment of the invention. The system 100 comprises a user device 101, a smell-user response database 102, and a smell history database 103. The user device is for example a mobile phone, smart phone, tablet, or some other portable or handheld user device. The user device 101 and the databases 102 and 103 are connected to each other through one or more communication networks (not shown). The communication networks may be formed of a plurality of interconnected networks or other communication systems including for example one or more of wireless local area network, wired networks, cellular and satellite communication networks. The user device 101 is configured to access the databases 102 and 103 through the communication networks.

In an embodiment, the smell-user response database 102 stores knowledge regarding possible user responses to specific smells. In an embodiment the database 102 is continuously updated as knowledge relating to possible responses evolves. The database can be updated by a service provider, or it can be open to other parties for providing updated information. In an embodiment the smell history database 103 is a database where user smell history with regard to smells encountered by the users (or user devices) at different locations/retailers are recorded, uploaded, and stored.

A skilled person understands that for the sake of clarity only one user device and one instance of each database type are shown in FIG. 1 and that in practice there may exist plurality of these, such as thousands or tens of thousands of these.

FIG. 1 shows an example where user device 101 detects ambient smell in 11 and initiates a smell analysis in the user device. This may be performed e.g. by a smell privacy application installed in the user device 101. In an example embodiment a smell impact is determined in 15 in response to detection 11 of the ambient smell. In an embodiment the smell impact value quantifies the impacts the smell may have on the user. In an example embodiment the smell impact value illustrates likely harmfulness of the detected ambient smell. In an embodiment the smell impact quantifies impact on privacy of the user.

In an example embodiment the user device 101 initiates a process to find out expected responses to the detected ambient smell. For this purpose the user device contacts the smell-user response database 102 and sends to the database 102 information about the detected ambient smell in 12. The database 102 responds with information about an expected user response in 13.

On the basis of the expected user response and/or other available information the user device 101 computes the smell impact value in 15. In an embodiment the smell impact is determined on the basis of one or more of the following or any combination thereof:
type of response the smell can generate in the user (expected user response)—its ability to influence user behavior,
user sensitivity to the expected user response,
other nearby smells, which may be deducted from the sensed ambient smell for determination of the smell impact,
malicious intent of the smell—degree of harmfulness of the ambient smell,
trustworthiness (or reliability and/or accuracy) of the scent source/generator,
(smell related) feedback provided by other users, and
history of smells—e.g. degree of change with respect to smell emanated by the same product/location/provider previously. The required information can be obtained for example from the database 103.

History of smells and feedback provided by other users may relate to quantifying the trustworthiness of a retailer. Determination of trustworthiness of the retailer may comprise comparing the user's observations with that of smells encountered by other users at that retailer. A crowdsourced solution is envisioned here, which requires comparing smell (and corresponding induced reactions) related data of multiple users. Also this is a task that cannot be performed without a technical automated solution especially in real time or with delays small enough not to inhibit useful implementation.

In an embodiment shown in FIG. 1 information about the history of smells and feedback provided by other users is stored in the smell history database 103. In an example implementation, for obtaining the history information the user device 101 contacts the smell history database 103, and sends to the database 103 information about the location associated with the detected ambient smell in 17. The database 103 responds with information relating to smells previously encountered by other users at that location possibly accompanied with feedback provided by other users in 18. In an alternative embodiment the history information may be stored in the user device 101.

A detailed example implementation for computing the smell impact value is discussed later in this document.

On the basis of the computed smell impact, the user device 101 determines an action that is to be taken in 16. In an embodiment, the action is a counter-measure for the detected ambient smell. That is, a counter action is determined and performed on the basis of the determined smell impact. The action may depend on the harmfulness of the detected ambient smell. In an embodiment, the counter action is a privacy preserving action.

The counter action can be for example one of the following or a combination thereof:
Recommending a new location to the user. E.g. a more “smell friendly” location (in the store) can be recommended.
Starting active protection for example by emitting a counter smell.
Notifying the user of the smell impact. The user can be notified of possible influences/consequences, too.

In an example embodiment the counter action is to do nothing. For example, if it is determined that the smell impact on the particular user in question is minimal, it is likely that the ambient smell does not have significant influence on the user, whereby there is no need for any actions. Also if it is determined that the smell impact would only increase by moving to any near location, the counter action may be to do nothing.

A detailed example implementation for determining the action to be taken is discussed later in this document.

FIG. 2 shows a block diagram of an apparatus according to an example embodiment of the invention. The apparatus can be for example the user device 101 of FIG. 1 or some other electronic device. The apparatus 101 comprises a communication interface module 250, a processor 240 coupled to the communication interface module 250, and a memory 260 coupled to the processor 240. The apparatus further comprises an input/output (I/O) unit 230, and a user interface (U/I) unit 110, such as a touch sensitive display, and a smell sensor 270, which are coupled to the processor 240. The apparatus may also comprise elements that act in more than one of said roles: for example, a camera or sensor may be configured to detect any of user's gestures, eye movements, facial movements, pulse, breathing frequency and/or steps taken by the user such that the same element can both act as a user interface input and as a sensor that reflects user response to ambient stimulus or stimuli.

It shall be understood that any coupling in this document refers to functional or operational coupling; there may be intervening components or circuitries in between coupled elements unless expressly otherwise described.

The memory 260 comprises a work memory and a non-volatile memory such as a read-only memory, flash memory, optical or magnetic memory. In the memory 260, typically at least initially in the non-volatile memory, there is stored software 270 operable to be loaded into and executed by the processor 240. The software 270 may comprise one or more software modules and can be in the form of a computer program product that is software stored in a memory medium. In the context of this document, a “memory medium” may be any non-transitory media or means that can contain, store, communicate, propagate or transport the instructions for use by or in connection with an instruction execution system, apparatus, or device, such as a computer.

The communication interface module 250 is configured for communication connections over one or more wired and/or wireless links. The communication interface 250 may implement telecommunication links suited for establishing links with other users or for data transfer, e.g. using the Internet. Such telecommunication links may be links using any of: wireless local area network links, Bluetooth, ultra-wideband, cellular or satellite communication links. The communication interface 250 may be integrated into the apparatus 100 or into an adapter or card that may be inserted into a suitable slot or port of the apparatus 100. While FIG. 2 shows one communication interface 250, the apparatus may comprise a plurality of communication interfaces 250. In a further example embodiment, the apparatus 100 further comprises a near field communication (NFC) unit.

The processor 240 is, for instance, a central processing unit (CPU), a microprocessor, a digital signal processor (DSP), a graphics processing unit, an application specific integrated circuit (ASIC), a field programmable gate array, a microcontroller or a combination of such elements. FIG. 2 shows one processor 240, but the apparatus 101 may comprise a plurality of processors.

As mentioned in the foregoing, the memory 260 may comprise volatile and a non-volatile memory, such as a read-only memory (ROM), a programmable read-only memory (PROM), erasable programmable read-only memory (EPROM), a random-access memory (RAM), a flash memory, a data disk, an optical storage, a magnetic storage or a smart card. In some example embodiments, only volatile or non-volatile memory is present in the apparatus 100. Moreover, in some example embodiments, the apparatus comprises a plurality of memories. In some example embodiments, various elements are integrated. For instance, the memory 260 can be constructed as a part of the apparatus 101 or inserted for example into a slot or a port. Further still, the memory 260 may serve the sole purpose of storing data, or it may be constructed as a part of an apparatus serving other purposes, such as processing data. Similar options are available also for various other elements.

The smell sensor 270 is configured to sense ambient smell and to convey information about the ambient smell for further processing in the processor 240. The smell sensor 270 can be for example an electronic nose.

A skilled person appreciates that in addition to the elements shown in FIG. 2, the apparatus 101 may comprise other elements, such as microphones, displays, as well as additional circuitry such as a camera unit, further input/output (I/O) circuitries, memory chips, application-specific integrated circuits (ASIC), processing circuitry for specific purposes such as source coding/decoding circuitry, channel coding/decoding circuitry and ciphering/deciphering circuitry. Additionally, the apparatus 101 may comprise a disposable or rechargeable battery (not shown) for powering the apparatus if external power supply is not available.

Still further, the apparatus 101 of FIG. 2 may comprise an element (not shown) that is configured to emit a counter smell in response to instructions provided by the processor 240. In yet another variation the apparatus 101 of FIG. 2 does not necessarily comprise the communication interface 250. In such case, the apparatus 101 is configured to operate as a stand-alone device that does not communicate with other devices.

It is also useful to realize that the term apparatus is used in this document with varying scope. In some of the broader claims and examples, the apparatus may refer to only a subset of the features presented in FIG. 2 or even be implemented without any one of the features of FIG. 2. In an example embodiment term apparatus refers to the processor 240.

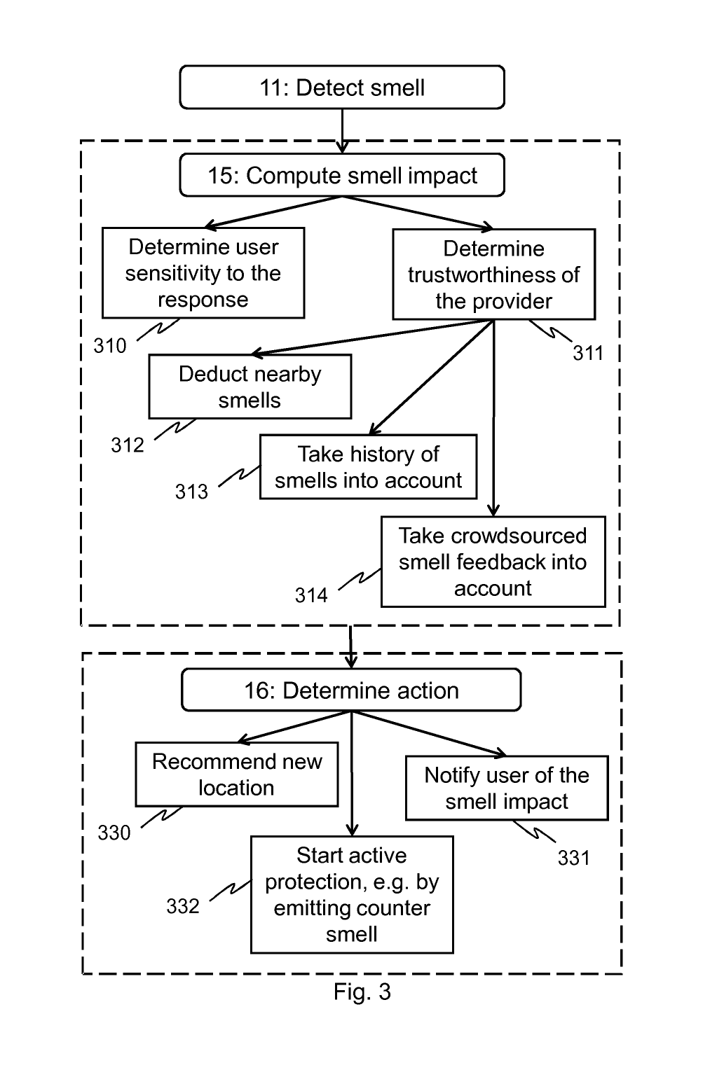

FIG. 3 shows a flow diagram illustrating a method according to an example embodiment of the invention. The method is performed for example in the user device 101 of FIGS. 1 and 2, such as a mobile phone, tablet or other handheld device.

In phase 11, ambient smell is detected. This may be performed for example by a smell sensor, such as an electronic nose.

In phase 15, smell impact is computed. Computing smell impact comprises one or more of the phases 310-314.

310: Determining user sensitivity to an expected response.

311: Determining trustworthiness of the provider of the smell.

312: Deducting nearby smells.

313: Taking history of smells into account.

314: Taking crowdsourced smell feedback into account.

In phase 16, an action to be taken is determined. This determination comprises one or more of the phases 330-332. In an embodiment, the action to be taken is determined only if the smell impact exceeds a predefined threshold value. Such predefined value may be a default value set by the system or a value defined by the user.

330: Recommending a new location/recommending changing location.

331: Notifying the user of the smell impact.

332: Starting/recommending active protection for example by emitting a counter smell.

In an example embodiment the following algorithm is used for dealing with and evaluating ambient smells:

1. Detecting Ambient Smell and Classifying its Type

A particular type of smell/odour is characterized by its mixture of comprising molecular compounds (which can be in hundreds). As such, the first step consists of detecting ambient smell, and classifying its type. Let st refer to the type of smell s. This detection and classification can e.g. be performed by a smell sensor in the user's device (e.g. sensor 270 of FIG. 2)—often referred to as the electronic nose.

2. Quantifying Smell Impact (e.g. Impact on Privacy)

The second step comprises computing the impact of the detected smell st with regard to the user profile.
Influence of the smell on the user—in terms of the types of responses (a particular type of smell) st can induce in the user. The types of responses range from getting implicit user feedback on a smell based product, e.g. perfumes; to actively manipulating the users' behaviour such that they spend more time in the stores, or “feel” more inclined to buy a product. Categorizing the smell influence on the user comprises one or more of the following steps:
   Public smell-response databases: The different types of smell, and the types of responses they can invoke in different users—is an area of active research. In an example embodiment the data corresponding to the mapping is stored in an updateable (public) database, denoted by D. The mapping is denoted as follows:

R({st₁,st₂, . . . }×{w₁,w₂, . . . }×{p₁,p₂, . . . })→{r₁,r₂, . . . }
where {p₁, p₂, . . . } refers to the different user profiles, {w₁, w₂, . . . } refers to the smell ‘amount’ (volume, density) the users are exposed to, and {r₁, r₂, . . . } refers to the responses induced by the corresponding smell types {st₁, st₂, . . . }.
Filter responses by user profile: While R gives the generic mapping, the actual response induced in a user u depends on his profile pᵤ, and the smell type stᵤ/amount wᵤ he has been exposed to. The filtering function ƒ is defined as follows:

$$f ( \frac{\{ {st}_{1} , {st}_{2} , … \}}{{st}_{u}} \times \frac{\{ {w}_{1} , {w}_{2} , … \}}{{w}_{u}} \times \frac{\{ {p}_{1} , {p}_{2} , … \}}{{p}_{u}} ) \to {r}_{u}$$

where rᵤ refers to the response that is likely to be induced in the user by the ambient smell.
Compute (weighted) smell impact:
   Sensitivity of the response: The same response exhibited by different users, can have different sensitivity values for them. For instance, while a person (man) might find his exhibition of a “favorable” response to a (ladies) perfume as particularly privacy sensitive; others might find the fact that they were influenced (by the ambient) smell to buy a specific brand of product as particularly privacy intrusive. Thus, in an example embodiment, the user's sensitivity level is mapped to the different responses—the response-sensitivity mapping function S is given below:

S({r₁,r₂, . . . })→{v₁,v₂, . . . }
where {v₁, v₂, . . . } refers to the corresponding user sensitivity levels. Given this, vᵤ refers to the user sensitivity to the anticipated response rᵤ in this case.

S(rᵤ)→vᵤ
Trustworthiness of the provider. The detected ambient smell is cross-referenced with the natural smell of the products being sold at the store/location, and the smell history at that location, to determine the malicious intent (if any) on behalf of the store provider. Necessary information about the natural smells and the smell history are obtained from a database that resides in a user device or in a database of a web service.
The “malicious intent” factor m_{q} of store provider q is thus computed as the type/amount of ‘unaccounted’ smell present in the store—which is basically the difference between the smell types/amounts {(st₁, w₁), (st₂, w₂), . . . } detected by the user device, and the expected smell types/amounts as a result of the nearby products/people in the store—denoted as {(st_{P1}, w_{P2}), (st_{P2}, w_{P2}), . . . }.
The computation is denoted below:

m_{q}=({(st₁,w₁),(st₂,w₂), . . . }−{(st_{P1},w_{P2}),(st_{P2},w_{P2}), . . . })×h_{q}
where h_{q} is a history scaling factor of provider q. In an embodiment the history scaling factor is increased each time an unaccounted smell is detected in the store—over the user's multiple visits to the store.
The computation can also take crowdsourced feedback into account, such that the experiences of other users—in terms of the smells/amounts detected during their visits to the store—while computing m_{q}. In an example embodiment time varying factors, e.g. the smells due to other people present in the store, or products whose smells evolve over time, are also taken into account while computing the unaccounted smell.
Smell impact (or privacy impact): Pl_{uq} of user u at provider q's store is finally computed as a product of

Pl_{uq}=vᵤ×m_{q}

3. Recommending a Counter Action (e.g. a Privacy Preserving Action)

In an example embodiment, once a malicious smell has been detected at a location, with smell impact Pl_{uq} greater than a threshold tᵤ (the threshold being user defined or a default value set by the system); the system determines and recommends the most suitable/effective counter action e.g. to protect user privacy. Example recommendations comprise:
Notification: Notify the user (in a user friendly fashion) that he is currently subject to a specific type of smell and its impact.
Move to a more smell friendly location in the store: The device continuously scans, and (re)computes the smell impact Pl_{uq} on the user, as the user moves in the store—to identify and recommend a location with less influencing ambient smells.
Active protection: Also active protection mechanisms can be used. The device can be configured to produce and emit a counter smell based on the prevalent smell quantification—such that the overall impact of the resulting (merged) smell is less than the specified threshold tᵤ.

Foregoing disclosure discusses embodiments relating to scenarios where a smell sensor in the user's device detects ambient smell at a location, computes smell impact of the ambient smell, and recommends an appropriate counter action to the user.

In yet another embodiment the ambient smell that is detected comprises smells emanated by the user. In this example, rather than scanning for ambient smells in the surroundings, the device is configured to detect smells emanated by the user and recommend counter actions to hide or anonymize the user-induced smells from other people and from possible smell detectors in range. That is, rather than protecting the user from privacy compromising external smells, this embodiment enables a user to hide his personal smells caused e.g. by medication that the user has applied, or he is carrying with himself. Also in this way privacy of the user is improved.

In such scenario, a smell privacy preserving solution executes in a similar manner to the examples discussed hereinbefore. Initially the system can detect the presence of a nearby smell sensor, possibly determining the actual distance between the user/sensor and the sensor capabilities, but this is not mandatory.
1. Scan smell(s) emanated by the user or by objects carried by the user. Corresponds to sensing ambient smells.
2. Determine whether there are sensitive smells, which need to be obfuscated. Corresponds to determining the smell impact.
3. Recommend (or execute) a counter action.
   Notification: Notify the user of the sensitive smell he may want to obfuscate.
   Move to a more “smell friendly” location: The device recommends a location that is out of the range of the smell sensor. For anonymization of the smell source, the device can recommend hiding in a place crowded with other people—such that the user cannot be identified as the source of the smell (emanated by the user).
   Active protection: The device can be configured emit a (new) smell—to counter the effects of the user's sensitive smell, or to reduce its intensity, such that the sensitive smell can no longer be detected by the smell sensor or other people.

In the following some example use cases are discussed:

Case 1: User U enters a store S. A sense enabled system P on his device detects ambient smell s. P determines the impact of s with regard to U as ‘Privacy Unsafe’ based on the observation that not only can
s influence the buying behaviour of U with regard to the products in S, but
as s is only capable of influencing people of a specific genetic profile, U's response to s also reveals information regarding U's genetic profile.
   Accordingly, P classifies the smell s as ‘Privacy Unsafe’ for U. It further stores ‘store S at location I’ in its database of untrusted locations/providers, such that U can be protected in future as well.

Case 2: User walks in to a furniture store. On venturing into the kitchen section, she smells freshly baked cake. The user's smartphone (smell privacy application thereof) also detects this ambient smell, and classifies the smell as a “marketing” smell based on its knowledge of “natural” smells expected in a furniture store. The smell privacy application notifies the user that the cake baking smell is basically a “marketing” smell, used by the retailer to influence her buying decision. The user may thus make an objective decision ignoring the cake smell.

Case 3: User walks in to a furniture store. On venturing into the kitchen section, she smells peppermint/cinnamon. The user's smartphone (smell privacy application thereof) also detects this ambient smell, and classifies the smell as a “marketing” smell based on its knowledge of “natural” smells expected in a furniture store. The smell privacy application notifies the user that the peppermint/cinnamon smell is basically a “marketing” smell, used by the retailer to attract her attention to specific products in the store. The smell privacy application recommends that the user should move out of the kitchen section to avoid the smell's influence.

This use case 3 is based on an ambient smell that is aiming at influencing user's memory/attention. Both cinnamon and peppermint scents can be considered smells that influence user's memory/attention.

Following use case 4 is taking personal profiling into account. The scents are even more targeted and customized according to the user's gender in this case.

Case 4: User walks in to a furniture store. On venturing into the kitchen section, she smells freshly baked vanilla flavored cake. The user's smartphone (smell privacy application thereof) also detects this ambient smell, and classifies the smell as a “marketing” smell based on its knowledge of
“natural” smells expected in a furniture store,
the gender of the user, and
the fact that vanilla is particularly effective as a “marketing” smell for females.

The smell privacy application notifies the user that the ambient smell is basically a “marketing” smell, used by the retailer to target customers of her gender. The smell privacy application recommends that the user should try to make an objective decision ignoring the ambient smell, or move to a different part of the store.

Without in any way limiting the scope, interpretation, or application of the claims appearing below, a technical effect of one or more of the example embodiments disclosed herein is improved privacy. Various embodiments of the invention for example provide an automated solution that helps users to detect ambient smells that may be intended to influence user behaviour. Such detection is typically not possible by use of human senses only. Another technical effect of one or more of the example embodiments disclosed herein is improved mechanism for detecting situations that potentially compromise privacy. Another technical effect of one or more of the example embodiments disclosed herein is improved user experience. With the use of an apparatus according to various embodiments user's become more aware of the surrounding environment and may be able to make informed decisions concerning the actions they take compared to situations where the user operates based on her own senses only.

Another technical effect of one or more of the example embodiments disclosed herein is a value add-on application/service for mobile platform providers. Another technical effect of one or more of the example embodiments disclosed herein is a plugin/component for smell related applications to improve their privacy rating/privacy related features. Another technical effect of one or more of the example embodiments disclosed herein is a standalone application that protects user privacy against ambient smell. Yet another technical effect of one or more of the example embodiments disclosed herein is the ability to automatically simultaneously obtain and process measurement data from plural different sources and to update and control operation of plural different user devices accordingly.

Embodiments of the present invention may be implemented in software, hardware, application logic or a combination of software, hardware and application logic. The software, application logic and/or hardware may reside on the user device or apparatus 101. In an example embodiment, the application logic, software or an instruction set is maintained on any one of various conventional computer-readable media. In the context of this document, a “computer-readable medium” may be any non-transitory media or means that can contain, store, communicate, propagate or transport the instructions for use by or in connection with an instruction execution system, apparatus, or device, such as a computer, with one example of a computer described and depicted in FIG. 2. A computer-readable medium may comprise a computer-readable storage medium that may be any media or means that can contain or store the instructions for use by or in connection with an instruction execution system, apparatus, or device, such as a computer.

If desired, the different functions discussed herein may be performed in a different order and/or concurrently with each other. Furthermore, if desired, one or more of the before-described functions may be optional or may be combined.

Although various aspects of the invention are set out in the independent claims, other aspects of the invention comprise other combinations of features from the described embodiments and/or the dependent claims with the features of the independent claims, and not solely the combinations explicitly set out in the claims.

It is also noted herein that while the foregoing describes example embodiments of the invention, these descriptions should not be viewed in a limiting sense. Rather, there are several variations and modifications which may be made without departing from the scope of the present invention as defined in the appended claims.

1-26. (canceled)

27. An apparatus, comprising:
a sensor configured to sense ambient smell; and
a processor configured, responsive to the sensor sensing the ambient smell,
to determine smell impact based on the ambient smell, and
to conduct at least one counter action based on the smell impact.

28. An apparatus according to claim 27, wherein the counter action comprises recommending a new location to a user of the apparatus.

29. An apparatus according to claim 27, wherein the counter action comprises notifying a user of the apparatus about the smell impact.

30. An apparatus according to claim 27, wherein the counter action comprises starting active protection.

31. An apparatus according to claim 30, wherein the processor is configured to perform the active protection by controlling the apparatus to emit a counter smell to compensate the ambient smell sensed by the sensor.

32. An apparatus according to claim 27, wherein the determination of the smell impact comprises quantifying a degree of harmfulness of the ambient smell.

33. An apparatus according to claim 32, wherein the processor is configured to quantify the degree of harmfulness of the ambient smell on the basis of one or more of the following: user sensitivity to the ambient smell, trustworthiness of the provider of the ambient smell, history of smells associated with a location where the ambient smell is sensed, and feedback obtained from other users.

34. An apparatus according to claim 27, wherein the determination of the smell impact comprises obtaining information about an expected user response to the sensed ambient smell.

35. An apparatus according to claim 34, wherein the processor is configured to determine user sensitivity to the expected user response.

36. An apparatus according to claim 34, wherein the processor is configured to give weight to the expected user response on the basis of one or more of the following: user sensitivity to the expected user response, a degree of harmfulness of the ambient smell, trustworthiness of the provider of the ambient smell, history of smells associated with a location where the ambient smell is sensed, and feedback obtained from other users.

37. An apparatus according to claim 27, wherein the processor is configured to deduct nearby smells from the sensed ambient smell.

38. An apparatus according to claim 27, wherein the determination of the smell impact comprises taking into account history of smells associated with a location where the ambient smell is sensed.

39. An apparatus according to claim 27, wherein the determination of the smell impact comprises taking into account smell feedback obtained from other users.

40. An apparatus according to claim 27, wherein the ambient smell is a smell emanated by a user of the apparatus.

41. An apparatus according to claim 27, wherein the apparatus is a portable user device.

42. A method, comprising:
sensing ambient smell; and
responsive to sensing the ambient smell,
determining smell impact based on the ambient smell, and
conducting at least one counter action based on the smell impact.

43. A method according to claim 42, wherein the counter action comprises one or more of the following: recommending a new location to a user, notifying a user of the smell impact, and starting active protection.

44. A method according to claim 43, wherein the active protection comprises emitting a counter smell to compensate the ambient smell.

45. A method according to claim 42, wherein determining smell impact comprises quantifying a degree of harmfulness of the ambient smell.

46. A non-transitory computer-readable medium encoded with instructions that, when executed by a computer, perform responsive to sensing ambient smell:
determining smell impact based on the ambient smell; and
conducting at least one counter action based on the smell impact.
